(19) United States
(12) Patent Application Publication
(10) Pub. No.: US 2026/0191668 A1
(43) Pub. Date: Jul. 9, 2026

(54) PROSTHETIC TRIAL DEVICE

(71) Applicant: LIMACORPORATE S.P.A., San Daniele del Friul (IT)

(72) Inventors: Alessia Colado GIMENO, Kiel (DE); Marco ZUCCHET, Pordenone (IT); Christoph FIEDLER, Diekhof (DE)

(21) Appl. No.: 19/131,792

(22) PCT Filed: Nov. 20, 2023
(86) PCT No.: PCT/IT2023/050257

§ 371 Date: May 21, 2025

(30) Foreign Application Priority Data
Nov. 22, 2022 (IT) 102022000024018

(51) Int. Cl. A61F 2/46; A61F 2/30; A61F 2/36

(52) U.S. Cl. CPC A61F 2/4684; A61F 2/30734; A61F 2/36; A61F 2002/30332; A61F 2002/30354; A61F 2002/30474; A61F 2002/30738

(57) ABSTRACT

Prosthetic trial device (10) comprising a proximal trial component (11) and an annular trial sleeve (12), wherein said proximal trial component (11) is associated with said trial sleeve (12) along a common longitudinal axis (X).

### FIELD OF THE INVENTION

The present invention concerns a prosthetic trial device which is used in surgical procedures of revision of femoral prosthetic implants to temporarily simulate the definitive prosthetic implant and allow the surgeon to evaluate the parameters for the optimal choice of the latter.

### BACKGROUND OF THE INVENTION

Surgical procedures of revision of femoral prosthetic implants are known, in which the first prosthetic implant has to be replaced with a revision prosthetic implant.

During this procedure, the surgeon first removes the prosthetic implant to be replaced, then distally inserts a new stem in the medullary canal of the femur, and then associates a prosthetic trial device with the new stem in order to identify the correct parameters for choosing the definitive one.

The prosthetic trial implant comprises a proximal trial component, or trial neck, to which there is connected the stem on one side and the femoral head on the opposite side, and a trial sleeve configured to axially accommodate the part of the neck connected to the stem.

The neck and the trial sleeve are available in different sizes and can be oriented in such a way as to define a desired reciprocal angular position, characterized by a certain angle of version, around a common longitudinal axis.

The sizes of the prosthetic implant and the angular position of the components is verified through a “trial reduction”, which is the surgical maneuver that allows to ascertain the correct movement of the joint. At this point, the prosthetic trial device is removed, and a definitive prosthetic implant having the same characteristics as the newly verified trial one is inserted.

There are prosthetic trial devices that, however, require additional measuring instruments to define the correct version angle. These instruments are particularly cumbersome and have to be positioned by the surgeon during the procedure, so they involve additional steps and a dilation of the intervention times.

Trial devices are also known that allow to adjust the reciprocal angular position between the trial neck and the trial sleeve in a discreet manner, but do not allow to perform the trial reduction because the components are not anatomical. In this case, the surgeon needs additional operating technique steps to verify that the size of the components and the version angle are correct, with a consequent dilation of operating times.

Some known devices for measuring the version angle in surgical procedures of revision of femoral prosthetic implants are described in documents US 2012/0259338 A1 and US 2011/0046745 A1.

There is therefore the need to perfect a prosthetic trial device that can overcome at least one of the disadvantages of the state of the art.

To do this, it is necessary to solve the technical problem of easily adjusting and clamping the trial neck and trial sleeve in a certain angular position.

In particular, one purpose of the present invention is to provide a prosthetic trial device that allows an easy angular adjustment between the trial neck and the trial sleeve, without using additional external measuring instruments, which can be extracted from the patient without losing the angular adjustment in order to allow replication with definitive components in “back table”.

Another purpose of the present invention is to provide such a prosthetic trial device that is also anatomical, that is, able to be used to perform the trial reduction.

Another purpose of the present invention is to provide a prosthetic trial device that is also modular, that is, it comprises interchangeable components which can be assembled in a simple manner to obtain a large number of size combinations.

The Applicant has devised, tested and embodied the present invention to overcome the shortcomings of the state of the art and to obtain these and other purposes and advantages.

### SUMMARY OF THE INVENTION

The present invention is set forth and characterized in the independent claim. The dependent claims describe other characteristics of the present invention or variants to the main inventive idea.

In accordance with the above purposes and to resolve the technical problem disclosed above in a new and original way, also achieving considerable advantages compared to the state of the prior art, a prosthetic trial device according to the present invention can be used during an operation of revision arthroplasty of a femoral prosthetic implant. Advantageously, the prosthetic trial device has an anatomical shape that allows it to be used during a trial reduction.

The prosthetic trial device comprises a proximal trial component and a trial sleeve with an annular shape, wherein the proximal trial component is associated with the trial sleeve along a common longitudinal axis.

The proximal trial component is configured to be associated, during use, with a stem able to be inserted in the medullary canal of a femur.

Moreover, a part of the proximal trial component is axially inserted in the trial sleeve and configured to accommodate a terminal end of the stem.

In accordance with one aspect of the present invention, the prosthetic trial device comprises positioning means configured to position the proximal trial component axially with respect to the trial sleeve.

The positioning means are also configured to position the proximal trial component angularly, in a continuous or discreet manner, in a plurality of version angular positions around the longitudinal axis.

The prosthetic trial device also comprises angular clamping means configured to clamp the proximal trial component in a determinate version angular position.

Both the positioning means as well as the angular clamping means are an integral part of the prosthetic trial device. Therefore, this prosthetic trial device is always ready for use, without a surgeon having to prepare additional external measuring and clamping instruments.

Moreover, both the positioning means and also the clamping means are integrated in the proximal trial component and in the trial sleeve, and are comprised in their overall dimensions. The prosthetic trial device is therefore particularly compact and has the same overall dimensions as a definitive prosthetic implant, while integrating all the positioning and angular clamping means useful for establishing the reciprocal positions between the components.

In accordance with another aspect of the present invention, the proximal trial component is advantageously able to be angularly positioned in a continuous manner with respect to the trial sleeve, or with respect to the stem. In other words, the version angular positions can be reached in a continuous manner without any predefined discrete action. This allows to extend the operating flexibility of the prosthetic trial device compared to similar devices of the state of the art.

According to a variant, the proximal trial component can be angularly positioned in a discrete manner with respect to the trial sleeve, or with respect to the stem.

In accordance with another aspect of the present invention, the proximal trial component comprises a connection body axially constrained to the trial sleeve by means of the positioning means.

In accordance with another aspect of the present invention, the connection body is conformed to be accommodated axially in the trial sleeve and to accommodate a terminal end of the stem.

In accordance with another aspect of the present invention, the positioning means comprise at least one elastic tab on the connection body and a circumferential groove on the trial sleeve.

In accordance with another aspect of the present invention, the positioning means are of the snap-fit type.

In accordance with another aspect of the present invention, the proximal trial component comprises an end body which projects transversely from the connection body and has an axial cavity in which the angular clamping means are contained. This makes the device particularly compact, without protruding and cumbersome parts.

In accordance with another aspect of the present invention, the end body has a protruding part which develops along a transverse axis which is inclined with respect to the longitudinal axis and in which the cavity is created.

In accordance with another aspect of the present invention, the angular clamping means comprise an actuator and a clamping element attached to the actuator and pivoted to the proximal trial component, in particular to the end body, being rotatable between a distanced position and a clamping position.

In accordance with another aspect of the present invention, the actuator can comprise a mobile element sliding in the cavity along the transverse axis and a threaded bushing engaged in a corresponding threaded portion of the mobile element.

In accordance with another aspect of the present invention, the proximal trial component is monolithic.

In accordance with another aspect of the present invention, the connection body is rotatably associated with the trial sleeve by means of the at least one elastic tab which is engaged freely in the circumferential groove. In particular, the connection body is rotatably inserted in the trial sleeve.

In accordance with another aspect of the present invention, the trial sleeve has an annular edge facing, during use, toward the proximal trial component and against which the clamping element abuts when it is in the clamping position.

In accordance with another aspect of the present invention, the annular edge develops angularly by 360° around the longitudinal axis.

In accordance with another aspect of the present invention, the annular edge has a smooth clamping surface.

In accordance with another aspect of the present invention, the prosthetic trial device comprises axial clamping means configured to make the proximal trial component and the stem temporarily integral.

In accordance with a variant of the present invention, the connection body and the end body can be distinct and separate bodies, able to be mechanically associated with each other in a removable manner. In this case:
the connection body is also angularly constrained to the trial sleeve by means of the positioning means, which also comprise a clamping pin on the connection body and a corresponding axial channel on the trial sleeve;
the end body is rotatably associated with the connection body by means of the angular positioning means, which comprise at least one additional elastic tab on the connection body and a circumferential groove on the end body; and
the connection body has an abutment edge facing, during use, toward the end body and against which the clamping element abuts when it is in the clamping position.

The end body is advantageously provided with a seating mating in shape with the connection body and in which a part of the connection body is accommodated.

The variant with the two distinct and separate connection and end bodies has the advantage of requiring a smaller number of components to obtain the same size variability compared to the embodiment in which the proximal trial component is monolithic.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a front view of a revision prosthetic implant;

FIGS. 2-3 are embodiments of the prosthetic trial device of the present invention in an application of use;

FIG. 4 is a view of the prosthetic trial device of FIG. 2 in which the components are separated;

FIG. 5 is a view of the prosthetic trial device of FIG. 2 in which the components are assembled;

FIG. 6 is a view of the prosthetic trial device of FIG. 3 in which the components are separated;

FIG. 7 is a view of the prosthetic trial device of FIG. 3 in which the components are assembled.

We must clarify that the phraseology and terminology used in the present description, as well as the figures in the attached drawings also in relation as to how described, have the sole function of better illustrating and explaining the present invention, their purpose being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

With reference to FIG. 1, a revision femoral prosthetic implant 100 comprises a stem 110 able to be inserted in the medullary canal of a femur, a substantially hemispherical shaped head 111 able to be inserted in a corresponding acetabular seating of the hip, a proximal component, or neck, 112 to which there is connected, on one side, the stem 110 and, on the opposite side, the head 111, and a sleeve 113 configured to accommodate the part of the neck 112 connected to the stem 110.

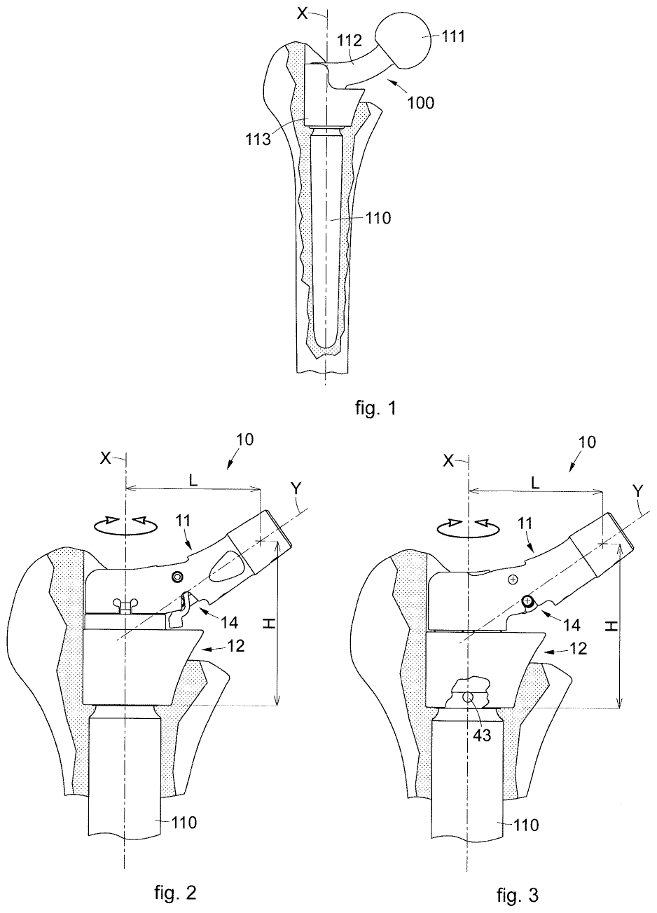

With reference to FIGS. 2-3, the prosthetic trial device 10 according to the present invention is able to be positioned, during use, on the previously implanted stem 110 to allow the surgeon to choose both the size of the definitive neck 112 and sleeve 113 as well as their reciprocal angular positioning in relation to the anatomical characteristics of the patient.

The prosthetic trial device 10 comprises a proximal trial component 11, hereafter referred to as a trial neck, and a trial sleeve 12.

The trial neck 11 and the trial sleeve 12 are separate and distinct components and are available in different sizes that can be combined with each other.

The trial neck 11 and the trial sleeve 12 have an anatomical shape similar to that of the corresponding definitive components. Therefore, the prosthetic device 10 can thus be used to perform a trial reduction prior to installing the definitive revision prosthetic implant 100.

The trial neck 11 is associated with the trial sleeve 12 along a common longitudinal axis X and can assume, with respect to the latter, a plurality of version angular positions.

As will become clearer in the following description, a part of the trial neck 11 is operatively inserted in the trial sleeve 12 along the longitudinal axis X.

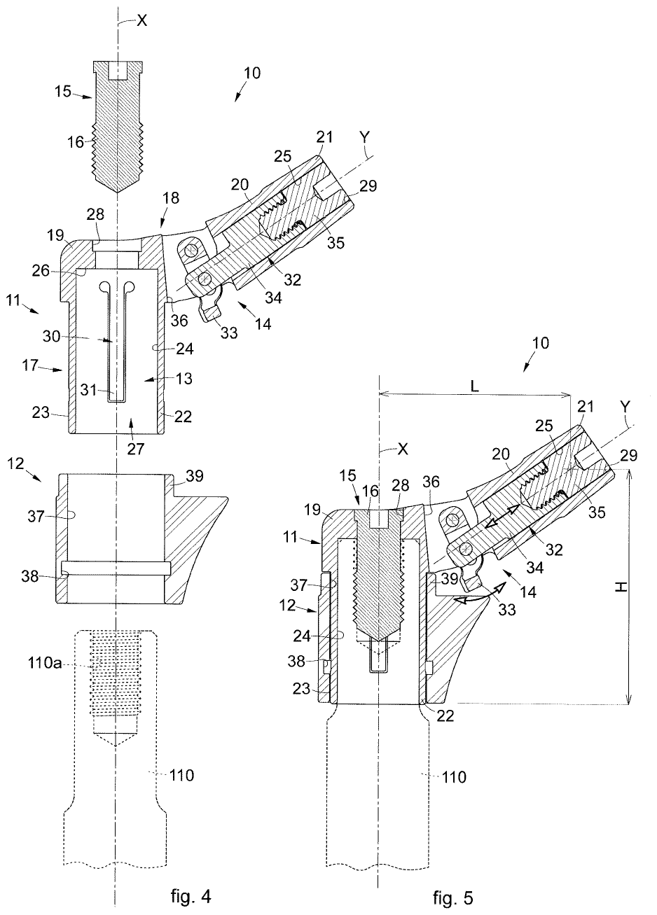

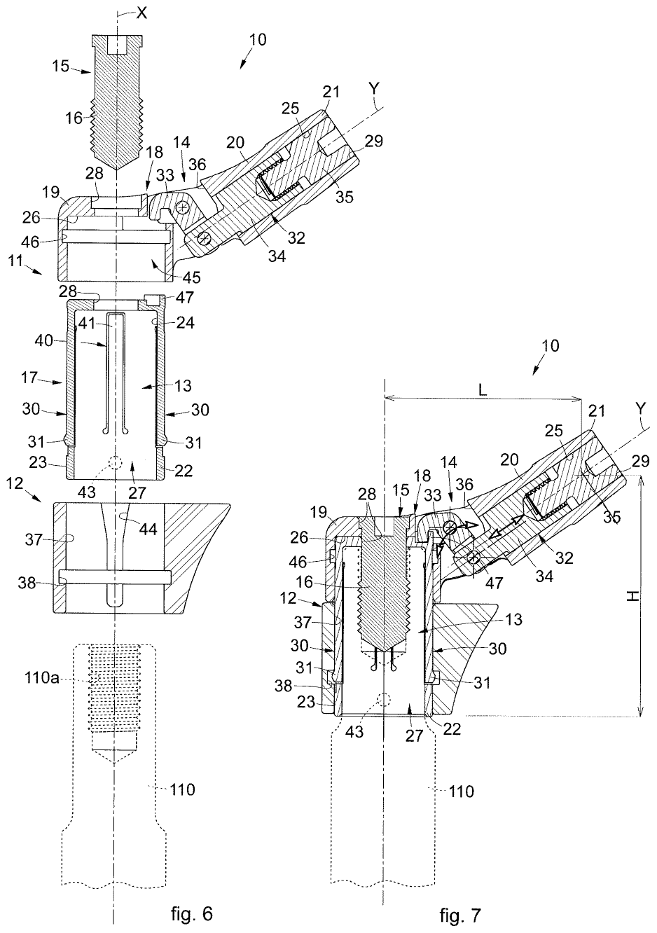

Referring to FIGS. 4-7, the prosthetic trial device 10 comprises positioning means 13 configured to position the trial neck 11 axially with respect to the trial sleeve 12.

According to some embodiments, the positioning means 13 are favorably configured to determine a snap-fit coupling between the trial neck 11 and trial sleeve 12. This greatly simplifies the coupling and reduces the number of components required, as well as constructively simplifying the prosthetic device 10.

The axial positioning between the trial neck 11 and the trial sleeve 12 is univocal, in the sense that an axial adjustment thereof is not provided.

The positioning means 13 are also configured to angularly position the trial neck 11 in a continuous manner with respect to the trial sleeve 12 in the plurality of version angular positions. This can occur essentially by rotating the trial neck 11 inserted in the trial sleeve 12 around the longitudinal axis X.

The prosthetic trial device 10 comprises angular clamping means 14 configured to clamp the trial neck 11 in the desired version angular position, as described in more detail below.

The prosthetic trial device 10 comprises axial clamping means 15 configured to make the prosthetic trial device 10 and the previously implanted stem 110 temporarily integral, in order to be able to perform the trial reduction. In the present case, the axial clamping means 15 comprise a surgical screw 16 which, during use, cooperates with a threaded portion 110a of the stem 110 and is able to be inserted along the longitudinal axis X.

The proximal trial component 11 comprises a tubular connection body 17 that develops along the longitudinal axis X and an end body 18 that projects transversely from the connection body 17. The angular adjustment of the prosthetic trial device 10 is functional to the angular positioning of the end body 18 in the plurality of version angular positions.

The end body 18 has a central part 19 coaxial to the longitudinal axis X and a protruding part 20 that develops along a transverse axis Y which is inclined with respect to the longitudinal axis X.

The protruding part 20 terminates in a shank 21, which can be cylindrical or frustoconical, and is configured to couple with the trial head 111.

The connection body 17 is conformed to couple both with the trial sleeve 12 and also with the previously implanted stem 110.

The connection body 17 is conformed to be axially accommodated in the trial sleeve 12. In turn, the connection body 17 is conformed to accommodate a terminal end of the stem 110.

Therefore, operationally, the temporary connection between the prosthetic trial device 10 and the stem 110 occurs between the latter and the proximal trial component 11.

This coupling mode advantageously allows to keep the parts that rotate “covered”, limiting any protruding elements that could create interference during the rotation movement. Moreover, the prosthetic trial device 10 is more compact and maneuverable, as well as less cumbersome.

In addition, the coupling thus obtained is similar to, if not exactly able to overlap with, the one that exists between the components of the definitive prosthesis that will be implanted. In this way, the trial reduction returns very faithful results compared to what can be expected once the definitive prosthesis has been implanted.

The connection body 17 has a substantially cylindrical wall 22 defining an external coupling surface 23 for the coupling with the trial sleeve 12 and an internal coupling surface 24 for the coupling with the stem 110.

The external coupling surface 23 terminates where the central part 19 of the end body 18 begins.

The internal coupling surface 24 defines, together with a bottom surface 26, a seating 27 coaxial to the longitudinal axis X and mating in shape with the proximal end of the stem 110. In the present case, the seating 27 has a cylindrical shape to allow an easy axial insertion/extraction of the stem 110.

The trial neck 11 has a through hole 28, coaxial to the longitudinal axis X and communicating with the seating 27 for the insertion of the surgical screw 16 which serves for the temporary clamping of the prosthetic trial device 10 with respect to the stem 110. Therefore, the seating 27 is axially open on two opposing sides: in correspondence with the hole 28, which passes through the bottom surface 26, and in correspondence with the access aperture to the seating 27 for the insertion of the stem 110.

Preferably the hole 28 has a transverse size, for example a diameter, smaller than a transverse size, for example a corresponding diameter, of the seating 27.

The connection body 17 also has at least one elastic tab, or strap, 30 carved into the wall 22 parallel to the longitudinal axis X and configured at least to define an axial constraint between the trial neck 11 and the trial sleeve 12, as will be described in more detail below. The elastic tab 30 is terminally provided with a tooth 31 that allows to determine the axial constraint.

The end body 18 has a cavity 25 which develops substantially along the transverse axis Y and in which the angular clamping means 14 are disposed. In other words, the angular clamping means 14 are disposed inside the trial neck 11, in particular in the protruding part 20 of the end body 18.

The cavity 25 is open outward, both axially in correspondence with the shank 21, where the end body 18 has an aperture 29, and also transversely in proximity to the connection body 17, where the end body 18 has at least one slot 36.

The angular clamping means 14 comprise an actuator 32 and a clamping element 33 attached to the actuator 32 and pivoted to the end body 18 in proximity to the central part 19. In the present case, the clamping element 33 is a lever provided with a free terminal part that has the function of determining the angular clamping.

The clamping element 33 is free to rotate and can possibly exit through the slot 36.

The actuator 32 comprises a mobile element 34 which is sliding in the cavity 25 along the transverse axis Y and a threaded bushing 35 engaged in a corresponding threaded portion of the mobile element 34.

The bushing 35 is positioned inside the cavity 25, in the part that corresponds to the shank 21, and it can be reached through the aperture 29.

The rotation of the bushing 35, for example by means of a wrench, a screwdriver or other suitable tool, causes the sliding of the mobile element 34 along the transverse axis Y and the consequent rotation of the clamping element 33 through the slot 36.

The clamping element 33 can rotate between a distanced position and a clamping position to fix the desired version angular position with respect to the trial sleeve 12 (FIGS. 2, 4-5) or with respect to the connection body 17 (FIGS. 3, 6-7).

The clamping element 33 has a shape such as to determine a clamping by friction between surfaces.

The trial sleeve 12 has an annular shape and is provided with a through hole 37 that develops along the longitudinal axis X and has a shape mating with the shape of the connection body 17, in particular with the shape of the wall 22 of the latter. In fact, the trial sleeve 12 is configured to axially receive the connection body 17.

According to some embodiments, the trial sleeve 12 advantageously has an asymmetrical shape with respect to the longitudinal axis X. In particular, the trial sleeve 12 has an eccentric portion facing the side of the protruding part 20 of the end body 18.

The trial sleeve 12 also has a circumferential groove 38 created internally on the wall that delimits the through hole 37. The circumferential groove 38 faces the through hole 37, has a continuous development all around the longitudinal axis X and is configured to receive the tooth 31 of at least one elastic tab 30.

The wall delimiting the through hole 37 has an essentially smooth surface. This essentially smooth surface is free of threads.

When the connection body 17 is inserted in the trial sleeve 12, the at least one elastic tab 30, which protrudes slightly from the wall 22 of the connection body 17, bends in contact with the wall of the through hole 37 until the tooth 31 engages the circumferential groove 38, determining the axial constraint between the trial neck 11 and the trial sleeve 12.

With particular reference to the embodiment of FIGS. 2 and 4-5, the trial neck 11 is monolithic, whereby the connection body 17 and the end body 18 are made in one piece.

The connection body 17 is rotatable with respect to the trial sleeve 12 by means of the angular positioning means 13 to angularly position the end body 18.

The angular positioning means 13 comprise the at least one elastic tab 30 and the circumferential groove 38. In the present case, the angular positioning means 13 comprise two diametrically opposing elastic tabs 30.

Operationally, the connection body 17, once inserted in the trial sleeve 12, can rotate around the longitudinal axis X guided by the sliding of the tooth 31 in the circumferential groove 38.

The angular clamping means 14 are configured to make the trial neck 11 temporarily integral with the trial sleeve 12 in a certain version angular position of the end body 18.

The trial sleeve 12 has an annular edge 39 facing, during use, toward the trial neck 11 and provided with an external contact surface against which the clamping element 33 abuts when it is taken in the clamping position. The annular edge 39 is substantially an extension of the wall that delimits the through hole 37.

The temporary coupling between the clamping element 33 and the annular edge 39 is obtained as a consequence of an essentially continuous angular positioning. In fact, advantageously, the annular edge 39 has a smooth surface, devoid of retentive references-for example grooves, indentations-whereby the clamping element 33 can be taken in any clamping position.

The annular edge 39 acts, at the upper part, as a stable rest for the trial neck 11. In particular, the wall 22 of the connection body 17 has a smaller transverse size than the transverse size of the central part 19 of the end body 18, whereby between the connection body 17 and the end body 18 there is a shoulder that acts as an end-of-travel for the trial neck 11 against the trial sleeve 12.

With particular reference to the embodiment of FIGS. 3 and 6-7, the trial neck 11 is made in two pieces, whereby the connection body 17 and the end body 18 are distinct and separate, and able to be associated with each other.

The connection body 17 is clamped both axially as well as angularly with respect to the trial sleeve 12.

The connection body 17 comprises at least one clamping pin 43 which projects radially from the wall 22 and is configured to engage in a corresponding axial channel 44 created in the wall which delimits the through hole 37 of the trial sleeve 12. The clamping pin 43 projects radially outward with respect to the seating 27 of the connection body 17.

The axial clamping is determined by the at least one elastic tab 30 in the manner previously described, while the angular clamping is determined by the geometric constraint that is established, during use, between the clamping pin 43 and the axial channel 44.

The end body 18 is associated with the connection body 17 in a rotatable manner to reach the desired version angular position.

The end body 18 is provided with a seating 45 mating in shape with the connection body 17 and in which the latter can engage to rotate.

The seating 45 is provided with a circumferential groove 46 created internally on the wall that delimits it. The circumferential groove 46 has a continuous development all around the longitudinal axis X.

Correspondingly, the connection body 17 comprises at least one additional elastic tab 40 carved into the wall 22 parallel to the longitudinal axis X, terminally provided with a tooth 41 and configured to define an axial clamping with the end body 18, engaging in the circumferential groove 46. This axial clamping occurs in a manner similar to what described previously.

The additional elastic tab 40 is oriented in the opposite direction with respect to the at least one elastic tab 30. In the present case, there are two elastic tabs 30 and two additional elastic tabs 40.

The angular positioning means 13 comprise the at least one additional elastic tab 40 and the circumferential groove 46.

In this case, the angular clamping means 14 are configured to make the end body 18 temporarily integral with the connection body 17.

For this purpose, the upper part of the connection body 17 is provided with an abutment edge 47, facing toward the protruding part 20 of the end body 18, on which the clamping element 33 can come into contact when it is taken in the clamping position.

According to a variant, if the trial neck 11 is made in two pieces, the end body 18 can be clamped both axially as well as angularly with respect to the connection body 17, while the connection body 17 can be rotatable with respect to the trial sleeve 12 and the version angular position is clamped by stopping the connection body 17 with respect to the trial sleeve 12.

The trial neck 11 is characterized by a height H, measured as the distance between a center point of the shank 21 and the base of the connection body 17, and by a lateral size, also called lateralization or offset, L measured as the distance between the center point of the shank 21 and the longitudinal axis X.

That said, the embodiment in which the trial neck 11 is monolithic is easier to make and manipulate; however, it requires a greater number of available trial necks 11 to obtain a certain size variability. For example, if the size variability includes three heights H1, H2, H3 and three lateralizations L1, L2, L3, it is necessary to have available nine components in order to obtain all the combinations.

On the contrary, the embodiment in which the trial neck 11 is modular, that is, made in two pieces, is more flexible and requires a smaller number of components to obtain the same size variability. Considering the previous example, six components would be sufficient to achieve the same size variability.

It is clear that modifications and/or additions of parts may be made to the prosthetic trial device 10 as described heretofore, without departing from the field and scope of the present invention, as defined by the claims.

It is also clear that, although the present invention has been described with reference to some specific examples, a person of skill in the art will be able to achieve other equivalent forms of a prosthetic trial device, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. A prosthetic trial device (10), configured to allow a trial reduction, comprising:
a proximal trial component (11) and an annular trial sleeve (12) associated with each other along a common longitudinal axis (X), said proximal trial component (11) being configured to be associated with a stem (110) which is able to be inserted in the medullary canal of a femur,
wherein it comprises both positioning means (13) configured to position said proximal trial component (11) axially with respect to said trial sleeve (12) and angularly in a plurality of version angular positions around said longitudinal axis (X), and also angular clamping means (14) configured to clamp said proximal trial component (11) in a certain version angular position, in which a part of said proximal trial component (11) is inserted axially in said trial sleeve (12) and configured to accommodate a terminal end of said stem (110).

2. The prosthetic trial device (10) as in claim 1, wherein said proximal trial component (11) is able to be angularly positioned in a continuous manner.

3. The prosthetic trial device (10) as in claim 1, wherein said proximal trial component (11) is able to be angularly positioned in a discrete manner.

4. The prosthetic trial device (10) as in claim 1, wherein said proximal trial component (11) comprises a connection body (17) axially constrained in said trial sleeve (12) by means of said positioning means (13).

5. The prosthetic trial device (10) as in claim 4, wherein said connection body (17) is conformed to be accommodated axially in said trial sleeve (12) and to accommodate a terminal end of said stem (110), and wherein said positioning means (13) comprise at least one elastic tab (30) on said connection body (17) and a circumferential groove (38) on said trial sleeve (12).

6. (canceled)

7. The prosthetic trial device (10) as in claim 1, wherein said positioning means (13) are of the snap-fit type.

8. The prosthetic trial device (10) as in claim 1, wherein said proximal trial component (11) comprises an end body (18) having an axial cavity (25) in which said angular clamping means (14) are contained.

9. The prosthetic trial device (10) as in claim 8, wherein said end body (18) has a protruding part (20) which develops along a transverse axis (Y) which is inclined with respect to said longitudinal axis (X) and in which said cavity (25) is created.

10. The prosthetic trial device (10) as in claim 9, wherein said angular clamping means (14) comprise an actuator (32) and a clamping element (33) attached to said actuator (32) and pivoted to said proximal trial component (11), being rotatable between a distanced position and a clamping position.

11. The prosthetic trial device (10) as in claim 10, wherein said actuator (32) comprises a mobile element (34) sliding in a cavity (25) along said a transverse axis (Y) and a threaded bushing (35) engaged in a corresponding threaded portion of said mobile element (34).

12. The trial device (10) as in claim 1, wherein said proximal trial component (11) is monolithic.

13. The prosthetic trial device (10) as in claim 5, wherein said connection body (17) is rotatably associated with said trial sleeve (12) by means of said at least one elastic tab (30) which is engaged freely in said circumferential groove (38).

14. The prosthetic trial device (10) as in claim 10, wherein said trial sleeve (12) has an annular edge (39) facing, during use, toward said proximal trial component (11) against which said clamping element (33) abuts when it is in the clamping position.

15. The prosthetic trial device (10) as in claim 14, wherein said annular edge (39) develops angularly by 360° around said longitudinal axis (X) and has a smooth clamping surface.

16. The prosthetic trial device (10) as in claim 4, wherein said connection body (17) and said end body (18) are distinct and separate bodies, able to be mechanically associated with each other in a removable manner.

17. The prosthetic trial device (10) as in claim 16, wherein said connection body (17) is also angularly constrained to said trial sleeve (12) by means of said positioning means (13), which comprise a clamping pin (43) on said connection body (17) and a corresponding axial channel on said trial sleeve (12).

18. The prosthetic trial device (10) as in claim 16, wherein said end body (18) is provided with a seating (45) mating in shape with said connection body (17) and in which a part of said connection body (17) is accommodated.

19. The prosthetic trial device (10) as in claim 16, wherein said end body (18) is rotatably associated with said connection body (17) by means of said angular positioning means (13), which comprise at least one additional elastic tab (40) on said connection body (17) and a circumferential groove (46) on said end body (18).

20. The prosthetic trial device (10) as in claim 10, wherein said connection body (17) has an abutment edge (47) facing, during use, toward said end body (18) and against which said clamping element (33) abuts when it is in the clamping position.

21. The prosthetic trial device (10) as in claim 1, wherein it comprises axial clamping means (15) configured to make said proximal trial component (11) and said stem (110) temporarily integral.
